(19) United States
(12) Patent Application Publication
(10) Pub. No.: US 2024/0139236 A1
(43) Pub. Date: May 2, 2024

(54) COPPER HISTIDINATE COMPOSITIONS AND USES THEREOF

(71) Applicant: Cyprium Therapeutics, Inc., New York, NY (US)

(72) Inventors: Lung S. Yam, New York, NY (US); Robert Niecestro, New York, NY (US); Shama Munim, New York, NY (US)

(21) Appl. No.: 18/280,113

(22) PCT Filed: Mar. 17, 2022
(86) PCT No.: PCT/US2022/020786

§ 371 Date: Sep. 1, 2023

Related U.S. Application Data
(60) Provisional application No. 63/162,975, filed on Mar. 18, 2021.

(51) Int. Cl. A61K 33/34; A61K 9/00; A61K 9/19; A61K 31/4172; A61P 3/02

(52) U.S. Cl. CPC A61K 33/34; A61K 9/0019; A61K 9/19; A61K 31/4172; A61P 3/02

(57) ABSTRACT

Menkes disease is a X-linked recessive disorder of brain copper metabolism caused by mutations in an essential mammalian copper transporter gene, ATP7A, affecting approximately 200 to 400 individuals in the United States. Copper replacement therapy has been used to treat Menkes disease. The present disclosure shows that treatment of Menkes disease. The present disclosure shows that treatment of Menkes disease patients with copper histidinate increases survival of the patients.

### CROSS-REFERENCE TO RELATED APPLICATIONS

This application claims benefit of priority under 35 U.S.C. § 119(e) of U.S. Ser. No. 63/162,975 filed Mar. 18, 2021, the entire contents of which is incorporated herein by reference in its entirety.

### BACKGROUND OF THE INVENTION

### Field of the Invention

The present invention relates generally to methods of increasing serum copper levels and more specifically to the treatment of Menkes Disease or copper deficiency in a subject by the administration of copper histidinate.

### Background Information

Menkes disease is a X-linked recessive disorder of brain copper metabolism caused by mutations in an essential mammalian copper transporter gene, ATP7A, affecting approximately 200 to 400 individuals in the United States. The estimated incidence rate is 1 in 100,000 live births. Untreated affected individuals suffer significant neurological symptoms such as seizures, hypotonia, failure to thrive, and neurodevelopmental delays that usually commence at 6 to 8 weeks of age. Death by age three years is a typical outcome of the disorder when untreated.

Over the past 25 years, it has been observed at close hand the dismal natural history of Menkes disease, a X-linked recessive disorder of copper transport caused by diverse mutations in a copper transporting ATPase, ATP7A. Infants who appear healthy at birth and for the first two months of life, begin to lose their early developmental milestones (smiling, lifting the head, following with the eyes), develop seizures, and begin an inexorable physical decline that usually results in premature mortality by three years of age.

Clinical and pathological features of this condition reflect deficiencies of enzymes that normally require copper as a cofactor. These include dopamine-beta-hydroxylase (DBH), cytochrome c oxidase (CCO), superoxide dismutase (SOD), lysyl oxidase (LO), and tyrosinase. Affected infants typically appear healthy at birth and advance normally for 3 to 4 months; however, subsequent neurodevelopment is arrested and loss of early milestones (smiling, visual tracking, head control, rolling over) is common. Cerebral and cerebellar atrophy, and dysmyelination occur, often accompanied by seizures. Additional clinical features include failure to thrive, and unusual connective tissue abnormalities (ascribed to LO deficiency): skin and joint laxity, urinary bladder diverticula, pectus excavatum, generalized vascular tortuosity, and multiple rib fractures. Decreased skin and hair pigmentation are often present, related to tyrosinase deficiency. In addition, the hair of symptomatic affected infants has a steel wool-like texture, and individual hair shafts show pili torti (180° twisting) when examined by light microscopy. The basis for this latter feature is uncertain and may involve a yet undescribed copper enzyme needed for keratin cross-linking.

Biochemical manifestations of Menkes disease include low concentrations of copper in plasma due to impaired intestinal absorption, and low liver and brain copper. However, certain other tissues (duodenal mucosa, kidney, spleen, pancreas, skeletal muscle, placenta) tend to accumulate copper in this disorder. The copper accumulation phenotype is also evident in cultured fibroblasts and lymphoblasts, in which excess retention of radiolabeled copper is demonstrable in pulse-chase experiments. This biochemical and cellular phenotype denotes a primary defect in copper egress and is consistent with evidence on the intracellular localization of the Menkes protein to both the trans-Golgi network and plasma membrane. Trans-Golgi localization helps to explain certain of the copper enzyme deficiencies in Menkes patients, e.g., DBH, LO, and tyrosinase are synthesized in the endoplasmic reticulum and processed through the trans-Golgi secretory pathway. Copper incorporation by the apoenzyme likely occurs during this processing. Re-localization of ATP7A to the plasma membrane after copper loading of normal cells indicates how copper exodus also is normally mediated by this gene product. More recent data indicate that ATP7A normally responds to N-methyl-D-aspartate receptor activation in the brain, and an impaired response likely contributes to the neuropathology of Menkes disease.

Several issues must be addressed in configuring therapeutic strategies for Menkes disease: (a) affected infants must be identified and treatment commenced very early in life before irreparable neurodegeneration occurs, (b) the block in intestinal absorption of copper must be bypassed, (c) circulating copper must be delivered to the brain, and (d) copper must be available to enzymes within cells that require it as a cofactor. The recent recognition that plasma catechol levels provide a reliable diagnostic marker for Menkes disease in otherwise asymptomatic newborns helps significantly with early recognition, and the primary transport block at the gastrointestinal level can be overcome by administering copper injections. However, the blood-brain barrier poses a more challenging obstacle, and delivery of copper to the brain with restoration of copper enzyme activities in brain remain significant obstacles. Since expression of the Menkes homolog has been demonstrated in rodent astrocytes, cells that contribute (with brain capillary endothelial cells) to the blood-brain barrier, it seems likely that transport of copper into the brain normally utilizes the Menkes transporter.

### SUMMARY OF THE INVENTION

The present invention is based on the seminal discovery that treatment of Menkes disease subjects with copper histidinate increases the survival of the subjects. Specifically, diagnosis and treatment of Menkes disease or other copper deficiencies in a subject within four weeks of birth (“early treatment”), or thereafter (“late treatment”) with copper histidinate results in an increase in survival of the subject. Copper histidinate is a pharmaceutical therapy that restores copper homeostasis and can be injected subcutaneously (SC) to bypass the defect in copper absorption through the gastrointestinal tract in Menkes disease patients or patients with copper deficiencies.

In one embodiment, the present invention provides a method of increasing survival, e.g., by at least about 50%, in a subject with Menkes disease by administering copper histidinate at a dose of about z to the subject. In one aspect, the subject is four weeks old or younger at initial administration. In one aspect, the subject is four weeks old or younger, less than one year old or greater than one year old. In another aspect, the administration is once or twice a day. In an additional aspect, the copper histidinate is administered at about 1450, once or twice a day. In a further aspect, the subject has a mutation in the ATP7A gene. In various aspects, the mutation in the ATP7A gene is a nonsense variant (stop gained), missense variant, intron variant, splice region variant, frameshift variant, splice acceptor variant, 5′ UTR variant, splice donor variant, duplication variant, in frame deletion, gross deletion, chromosomal translocation, or a combination thereof. In one aspect, survival is increased by at least about 50%, 55%, 60%, 65%, 70%, 75%, 80%, 85%, 90%, 95% or 100%. In an additional aspect, increased survival is by at least about 1, 2, 3, 4, 5, 6, 7, 8, 9 10, 11, 12, 13, 14, 15, 16, 17, 18 or more years.

In an additional embodiment, the present invention provides a method of increasing serum copper levels in a subject by administering copper histidinate at a dose of about 1000-8900 μg/day to the subject, wherein the subject is four weeks old or younger at initial administration, thereby increasing serum copper levels. In one aspect, the subject is four weeks old or younger, less than one year old or greater than one year old. In another aspect, the administration is once or twice a day. In an additional aspect, the copper histidinate is administered at about 1450 μg/day for once-a-day dosing and 2900 μg/day for twice a day dosing. In a further aspect, the administration is by subcutaneous injection. In one aspect, the subject has Menkes disease. In a further aspect, the subject has a mutation in the ATP7A gene. In various aspects, the mutation in the ATP7A gene is a nonsense variant (stop gained) missense variant, intron variant, splice region variant, frameshift variant, splice acceptor variant, 5′ UTR variant, splice donor variant, duplicative variant in frame deletion, gross deletion, chromosomal translocation or a combination thereof. In certain aspects, the serum copper, serum ceruloplasmin, serum bicarbonate, serum creatinine and/or urine beta-2 microglobulin levels of the subject are monitored. In various aspects, the serum copper level of the subject prior to administration of the copper histidinate is less than about 75 ug/dl. In one aspect, following administration of copper histidinate, serum copper level of the patient will be at least about 85 μg/dl.

In a further embodiment, the present invention provides a pharmaceutical composition of lyophilized copper histidinate and a pharmaceutically acceptable carrier. In one aspect, the pharmaceutically acceptable carrier is saline. In an additional aspect, the lyophilized copper histidinate is reconstituted with saline. In a further aspect, the reconstituted copper histidinate has a concentration of about 2900 μg/ml.

In one embodiment, the present invention provides a stable lyophilized pharmaceutical formulation of copper histidinate, wherein the formulation made by lyophilizing an aqueous solution comprising 2 moles of L-histidine and 1 mole of cupric chloride and adjusted to a pH of about 7.0-7.5, for example, 7.35, in an aqueous solution, followed by lyophilization of the solution. In one aspect, the copper histidinate is present at about 2900 μg/mL in the aqueous solution. In an additional aspect, the formulation is stable for greater than two months and preferably for at least about 18 months.

In an additional embodiment, the present invention provides a vial comprising a formulation of copper histidinate and a pharmaceutically acceptable carrier, wherein the formulation is made by lyophilizing an aqueous solution comprising 2 moles of L-histidine and 1 mole of cupric chloride and adjusted to a pH of about 7.0 to 7.5 in an aqueous solution, followed by lyophilization of the solution. In one aspect the pH is about 7.35. In one aspect, the pharmaceutically acceptable carrier is saline. In an additional aspect, the formulation is reconstituted in 2 ml of saline to a concentration of about 2900 μg/ml. In a further aspect, the formulation comprises lyophilized copper histidinate reconstituted in saline at a concentration of 500 μg elemental copper/ml in the vial. In certain aspects, the vial further comprises instructions for reconstitution of the lyophilized copper histidinate in saline and subcutaneous administration to a subject with Menkes disease at a dose of 0.5 ml (or cc) per administration twice daily (1450 μg) until the subject is one year of age and once daily after the subject is one year of age. In one aspect, the lyophilized copper histidinate is stable at room temperature for at least 24 hours or at about 2-8° C. for at least about 7 days, 2 weeks, one month, two months or greater than two months. In certain aspects, the lyophilized copper histidinate is stable at room temperature for at least 24 hours or at about 4° C. In certain aspects, the lyophilized formulation is stable for about 18 months at about −10 to −20° C., 2 to 8° C., or room temperature (15 to 30° C.). In certain aspects, the subject with Menkes has an ATP7A mutation selected from a nonsense variant, missense variant, intron variant, synonymous variant, splice region variant, frameshift variant, splice acceptor variant, 5′ UTR variant, splice donor variant, duplicative variant, in frame deletion, gross deletion, chromosomal translocation, canonical splice site mutation or a combination thereof.

In another embodiment, a method of increasing serum copper levels in a subject by administering copper histidinate to the subject via a transdermal route of administration is provided. By way of example, the copper histidinate is incorporated within a cream, gel, powder, lotion, ointment, liniment, suspension, microemulsion, nanoemulsion, and/or liposome. In a preferred aspect, the route of administration is by a transdermal patch. In one aspect, the transdermal patch includes cyclodextrin.

In some aspects, the transdermal patch includes one or more transdermal permeabilizing agents. In one aspect, the amount of copper delivered transdermally is equivalent to about 100 μg to 3000 μg elemental copper. In one aspect, the amount of copper delivered is selected from about 100 μg elemental copper, 250 μg elemental copper; 500 μg elemental copper, 1000 μg elemental copper; 1500 μg elemental copper, 2000 μg elemental copper, 2500 μg elemental copper or 3000 μg elemental copper. In one aspect, the dosage is from about 1000 to 8900 μg copper histidinate per day; for example 1450 or 2900 μg copper histidinate per day.

In another embodiment, the invention provides a transdermal dosage form comprising copper histidinate and a pharmaceutically acceptable carrier. In one aspect, the dosage form includes copper histidinate present at a concentration sufficient to deliver from about 100 μg elemental copper, 250 μg elemental copper; 500 μg elemental copper, 1000 μg elemental copper; 1500 μg elemental copper, 2000 μg elemental copper, 2500 μg elemental copper or 3000 μg elemental copper. In one aspect, the transdermal dosage form is a transdermal patch. In one aspect, the dosage is from about 1000 to 8900 μg copper histidinate per day; for example 1450 or 2900 μg copper histidinate per day.

In yet another embodiment, the invention provides a container with two chambers for mixing a powdered medicament, in this instance lyophilized copper histidinate, with a diluent such as saline, prior to injecting the mixed ingredients into a subject through a needle externally affixed to the container. The container can be a syringe, a vial, an ampoule or the like.

In another embodiment, the present disclosure provides a method of increasing serum copper histidinate levels in a subject including administering copper histidinate to the subject via a subcutaneous route of administration with a peak detectable level of copper histidinate in the serum at about ½ hour to 1 hour following administration, thereby increasing serum copper histidinate levels in the subject. In one aspect, the subject has a copper deficiency, such as Menkes disease. In one aspect, the copper histidinate distribution phase half-life is about 2-3 hours. In one aspect, the terminal elimination phase half-life t ½ is at about 30-150 hours. In one aspect, the terminal elimination phase half-life t ½ is about 75 hours. In one aspect, the subject is four weeks old or younger at initial administration. In one aspect, administration is about 100 μg elemental copper, 250 μg elemental copper; 500 μg elemental copper, 1000 μg elemental copper; 1500 μg elemental copper, 2000 μg elemental copper, 2500 μg elemental copper, 3000 μg elemental copper, 3500 μg elemental copper, 4000 μg elemental copper, 4500 μg elemental copper, or 5000 μg elemental copper. In another aspect, administration of copper histidinate is about 1450 μg twice per day when the subject is less than one year old and about 1450 μg once per day when the subject is over one year old.

### BRIEF DESCRIPTION OF THE DRAWINGS

FIG. 1 shows the arithmetic mean whole blood and serum total radioactivity and serum [¹⁴C]-copper histidinate concentration-time profiles following a single subcutaneous bolus dose of 3.0 mg (˜500 nCi) [¹⁴C]-Copper Histidinate in healthy adult male subjects (semi-log scale) (pharmacokinetic population).

### DETAILED DESCRIPTION OF THE INVENTION

The present invention is based on the seminal discovery that early treatment of Menkes disease subjects with copper histidinate increases the survival of the subjects. In one aspect, treatment of Menkes disease patients within four weeks of birth with copper histidinate results in a significant increase in median overall survival, although treatment after 4 weeks of birth also results in an increase in survival in patients with copper deficiency.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Menkes disease is a neurodegenerative disease with X-linked recessive inheritance. Orally administered copper accumulates in the intestine, resulting in the failure of copper absorption. The primary metabolic defect that causes copper accumulation in the intestine is present in almost all extrahepatic tissues. The blood, liver and brain are in a state of copper deficiency, which is due to defective copper absorption. As such, in one aspect, the copper histidinate is administered by intravenous administration or subcutaneous administration. In one embodiment, the present invention provides a method of increasing survival by at least about 50% in a subject with Menkes disease by administering copper histidinate at a dose of about 1450-8900 μg to the subject, wherein the subject is four weeks old or younger at initial administration. In one aspect, the subject is four weeks old or younger, less than one year old or greater than one year old. In another aspect, the administration is once or twice a day. In an additional aspect, the copper histidinate is administered at about 1450 μg/twice a day when the subject is less than 12 months old and 1450 μg/per day when the subject is greater than one year old. In a further aspect, the subject has a mutation in the ATP7A gene. In various aspects, the mutation in the ATP7A gene is a nonsense variant (e.g., stop gained), missense variant, intron variant, splice region variant, frameshift variant, splice acceptor variant, 5′ UTR variant, splice donor variant, duplicative variant, in frame deletion, gross deletion, chromosomal translocation or a combination thereof. In one aspect, survival is increased by at least about 50%, 55%, 60%, 65%, 70%, 75%, 80%, 85%, 90%, 95% or 100%. In an additional aspect, increased survival is by at least about 1, 2, 3, 4, 5, 6, 7, 8, 9, 10, 11, 12, 13, 14, 15, 16, 17, 18 or more years.

Menkes Disease is a genetic disorder affecting the metabolism and/or transport of copper. Patients with this disease are physically and/or mentally disabled. The onset of Menkes disease usually occurs in the first 2-3 months of life. Infants born with the disease fail to thrive, experience hypothermia, have delayed development, and experience seizures. These infants also have characteristic physical features such as changes of their hair and face. Female patients may also have changes in hair and skin color.

In one aspect, the early diagnosis and treatment of Menkes disease increases the likelihood of survival for the subject. Menkes disease is typically diagnosed based on the clinical features, medical examination, and genetic testing for alterations in the ATP7A gene. Clinical features include neurological symptoms such as seizures, abnormal palate morphology, aplasia/hypoplasia of the abdominal wall musculature, developments regression, dilatation and dry skin. Other types of tests that may be helpful include analysis of catecholamines and copper levels in the blood. In one aspect, the subject is diagnosed with Menkes disease at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 days or more postnatal. In another aspect, the subject is diagnosed with Menkes disease at least about 1, 2, 3, 4, 5, 6, 7, 8, 9, 10, 11, 12, 13, 14, 15, 16, 17, 18, 19, 20, 21, 22, 23 or 24 or more weeks postnatal. In a further aspect, the subject is diagnosed with Menkes disease at least about 0.5, 1.0, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 18, 24, 30 or 36 months or more of age.

Appropriate treatment of Menkes Disease requires that the disease be diagnosed early and treatment started before irreversible brain damage occurs. The first year of life is especially critical for brain development. The aim of treatment is to bypass the normal route of absorption of copper through the gastrointestinal tract. Copper must then be delivered to brain cells and be available for use by enzymes. In an additional aspect, the subject is administered an initial dose of copper histidinate at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90 or 100 days or more postnatal. In a further aspect, the subject is administered an initial dose of copper histidinate at about one week, two weeks, three weeks, four weeks, five weeks, six weeks, seven weeks, eight weeks, nice weeks, ten week, eleven weeks, twelve weeks postnatal. In a further aspect, the subject is administered an initial dose of copper histidinate at about 0.5, 1.0, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 18, 24, 30 or 36 months or more of age. In one aspect, the subject is administered copper histidinate for about at least 0.1, 0.2, 0.3, 0.4, 0.5, 1, 1.5, 2, 2.5, 3, 4, 5, 6, 7, 8, 9, 10 or more 10 years. In an additional aspect, the subject is administered two doses of copper histidinate daily. In certain aspects, the subject is administered two doses of copper histidinate daily for the first 12 months of age. In a further aspect the subject is administered one dose of copper histidinate daily. By way of example, prior to one year of age, a subject is administered about 1450 μg copper histidinate, twice daily, or about 2900 μg per day and once the subject is about one year of age, administration is 1450 μg copper histidinate daily.

Copper histidinate, the active substance in the pharmaceutical compositions described herein, has an approximate pH of 7.0-7.5. The molecular formula is C₁₂H₁₈CuN₆O₄ with a molecular weight of 373.86 g/mol. In contrast, copper histidinate can be injected directly into the body to avoid absorption through the gastrointestinal tract. However, studies have shown the genetic abnormalities causing Menkes disease cannot simply be corrected by copper replacement injections. In the studies described herein, 1450 μg copper histidinate is equivalent to 250 μg elemental copper; 5800 μg copper histidinate is equivalent to about 1000 μg elemental copper; 8850 μg copper histidinate is equivalent to about 1500 μg elemental copper.

As used herein, the terms “survival” or “overall survival” are used interchangeably and refer to the length of time of onset from either the date of birth, date of diagnosis or the start of treatment for a disease, such as Menkes disease, that patients diagnosed with the disease are still alive. In a clinical trial, measuring the overall survival is one way to see how well a new treatment works. Specifically, increased survival refers the increased length of time a patient received copper histidinate treatment is alive compared to a Menkes patient who did not receive copper histidinate. Median survival is the amount of time after which 50% of the patients in a cohort have died and 50% have survived.

In certain aspects, survival is increased by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%. In additional aspects, increased survival is at least about. 1, 2, 3, 4, 5, 6, 7, 8, 9, 10, 11, 12, 13, 14, 15, 16, 17, 18 or more years.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, sub arachnoid, intraspinal and intrasternal, oral, sublingual buccal, rectal, vaginal, nasal ocular administrations, as well infusion, inhalation, and nebulization. In one aspect, administration is by subcutaneous administration.

The dose and frequency of copper histidinate that is administered depends on the age of the subject. In one aspect, the subject is under one year old and is administered copper histidinate twice a day. In an additional aspect, the subject is <12 months old and is administered copper histidinate twice a day. In an additional aspect, the subject is over one year old and is administered copper histidinate once a day. In a further aspect, the subject is >12 months old and is administered copper histidinate once a day. In certain aspects, the patient is administered 1450-8900 μg of copper histidinate. In specific aspects, the subject is administered 1450 μg copper histidinate daily. In another aspect, the subject is administered 5800 μg copper histidinate daily. In in a further aspect, the subject is administered 8850 μg copper histidinate daily. In some aspects, the subject is administered copper histidinate in the amount equivalent to 250 μg elemental copper; 1000 μg elemental copper; or 1500 μg elemental copper daily.

Transdermal or transmucosal drug delivery is an attractive route of drug delivery for several reasons. Gastrointestinal drug degradation and the hepatic first-pass effect are avoided. In addition, transdermal and transmucosal drug delivery is well-suited to controlled, sustained delivery (see, e.g., Elias, In Percutaneous Absorption: Mechanisms-Methodology-Drug Delivery, Bronaugh & Maibach, Eds., pp 1-12, Marcel Dekker, New York, 1989.). For many applications, traditional methods of administering drugs are not optimal because of the very large initial concentration of the drug. Transdermal delivery could allow a more uniform, slower rate of delivery of a drug. Moreover, patient compliance is encouraged because such delivery methods are easy to use, comfortable, convenient and non-invasive.

Several methods have been proposed to enhance transdermal transport of drugs. For example, chemical enhancers (Burnette, R. R. In Developmental Issues and Research Initiatives; Hadgraft J., Ed., Marcel Dekker: 1989; pp. 247-288), iontophoresis, and others have been used.

Typically iontophoresis is performed by placing an electrode containing an ionic drug solution in contact with the skin at a location where the drug is to be transported. A second electrode is placed on the skin near the first electrode, and voltage is applied so as to cause current to pass through the skin, thereby completing the electrical circuit between the electrodes. As current flows, the ionic drug molecules migrate through the skin under the influence of the second electrode. One general class of electrode designs involves the use of a conductive element associated with a compartment or pouch into which a drug solution is introduced. One wall of the pouch typically comprises a permeable barrier, which serves to contain the solution, but permits drug ions to pass there through. Examples of such electrodes can be seen in U.S. Pat. Nos. 4,250,878, 4,419,092, and 4,477,971, among others.

A second class of electrode designs involves the use of a conductive element associated with a gel material for containing ionized drug without the use of a pouch. Examples of such bioelectrodes are found in U.S. Pat. Nos. 4,383,529, 4,474,570, and 4,747,819. Typically, these gel-type electrodes incorporate ionized drug into the gel at the time of manufacture.

A third type of electrode designs generally utilizes a conductive element associated with a hydratable element. The hydratable element is typically formed of a stack of sheets of a dry cross-linked hydrogel such as cross-linked polyethylene oxide (PEO). U.S. Pat. Nos. 6,169,920 and 6,317,629 assigned to Alza disclose iontophoretic drug delivery devices. U.S. Pat. Nos. 5,087,242, 5,374,241, 5,730,716, 6,731,977 assigned to Iomed disclose electrodes and devices for iontophoretic delivery of agents. U.S. Pat. No. 5,681,580 assigned to Samsung Electro-Mechanics Co. discloses a patch-type device for iontophoretic transdermal medication of insulin. A different type of transdermal delivery device is disclosed in U.S. Pat. No. 6,148,232 to Avrahami. The device includes a plurality of electrodes, which are applied at respective points on skin of a subject, and a power source that applies electrical energy between two or more of the electrodes to cause ablation of the stratum corneum, primarily beneath the respective electrodes, and to generate micro-channels. Various techniques for limiting ablation to the stratum corneum are described, including spacing of the electrodes and monitoring the electrical resistance of skin between adjacent electrodes. Sintov et al. (J. Controlled Release 89: 311-320, 2003) and U.S. Pat. Nos. 6,597,946; 6,611,706; 6,708,060; and U.S. Pat. No. 6,711,435 to Avrahami disclose improvements and additional devices for ablating the stratum corneum and generating micro-channels so as to facilitate transdermal passage of substances through the skin. The devices are aimed at reducing sensation and minimizing damage to skin underlying the stratum corneum during micro-channel generation.

During passive transdermal delivery, the active agent is delivered into the mammal using a concentration gradient across the barrier (e.g., by manual diffusion through the skin). For example, patches containing high concentrations of drugs can be attached to the skin of a patient. Electricity may be used to facilitate drug transport through the skin barrier. In electrical aids, a potential (voltage) is applied to the membrane to facilitate drug transport. In transdermal ion osmotherapy, ionized drugs are transferred into the skin by an applied potential gradient. Anionic drugs are delivered into the skin under a negative electrode (negatively charged electrode), while cationic drugs are delivered under a positive electrode (positively charged electrode). Ion osmotherapy can enhance the permeability of ionic materials into the skin and can be better controlled. The most common design of an iontophoretic device includes a power source (e.g., a battery), an electrical control mechanism, and two separate conductive electrodes.

According to the present invention, transdermal delivery of copper histidinate may be accomplished in any of a variety of formats. In some embodiments, a nanoparticle or other composition comprising copper histidinate is incorporated within a cream, gel, powder, or lotion such that the copper histidinate is administered to a subject by application to the skin. In some embodiments, a composition comprising copper histidinate is incorporated within an ointment and/or liniment such that the therapeutic agent(s) are administered to a subject by application to the skin. In some embodiments, a composition comprising copper histidinate is incorporated within a suspension, microemulsion, nanoemulsion, and/or liposome such that the copper histidinate is administered to a subject by application to the skin. In some embodiments, a composition is incorporated within a transdermal patch such that copper histidinate is administered to a subject from the patch. Embodiments of the invention also provide for a patch for the transdermal delivery of compositions comprising a therapeutically effective amount of copper histidinate. In one embodiment, the invention provides a patch for the transdermal delivery of copper histidinate comprising a therapeutically effective amount of copper histidinate, wherein the composition further comprises a cyclodextrin.

In certain embodiments, a patch of the present invention further comprises a copper histidinate-containing composition that comprises one or more transdermal permeabilizing agents. Thus, in various embodiments, the invention provides a patch for the transdermal delivery of a pharmaceutical composition of the present invention that comprises a therapeutically effective amount of copper from copper histidinate. Further, in these and related embodiments the patch can be configured for the iontophoretic transdermal delivery of various pharmaceutical compositions and may include an electrode and a reservoir of the pharmaceutical composition. In some embodiments, the amount of copper delivered transdermally is equivalent to about 100 μg to 3000 μg elemental copper; for example, 100 μg elemental copper, 250 μg elemental copper; 500 μg elemental copper, 1000 μg elemental copper; 1500 μg elemental copper, 2000 μg elemental copper, 2500 μg elemental copper or 3000 μg elemental copper.

Menkes disease is caused by pathogenic mutations of the ATP7A gene. The ATP7A gene provides instructions for making a copper transporter protein that is important for regulating copper levels in the body. Copper is necessary for many cellular functions, but it is toxic when present in excessive amounts. The ATP7A protein is found throughout the body, except in liver cells. In the small intestine, this protein helps control the absorption of copper from food. In other cells, the ATP7A protein has a dual role and shuttles between two cellular locations. The protein normally resides in a cell structure called the Golgi apparatus, which modifies newly produced proteins, including enzymes. In the Golgi apparatus, the ATP7A protein supplies copper to certain enzymes that are critical for the structure and function of bone, skin, hair, blood vessels, and the nervous system. If copper levels in the cell environment are elevated, however, the ATP7A protein moves to the cell membrane and eliminates excess copper from the cell.

A gene mutation is a permanent alteration in the DNA sequence that makes up a gene, such that the sequence differs from what is found in most people. Mutations range in size; they can affect anywhere from a single DNA building block (base pair) to a large segment of a chromosome that includes multiple genes. Examples of types of gene mutations include missense variant, intron variant, splice region variant, frameshift variant, splice acceptor variant, 5′ UTR variant, splice donor variant, nonsense (stop gained), in frame deletion, gross deletion, duplication, and chromosomal translocation.

A missense mutation or variant is a point mutation in which a single nucleotide change results in a codon that codes for a different amino acid residue in a protein sequence.

An intron mutation or variant is a mutation that occurs with the intron region of the gene. Introns occupy about 40% on average of the total length of genes, which means that most randomly occurring mutations will fall into intron regions, and do not affect protein sequences and functions.

A splice region mutation or variant is a genetic alteration in the DNA sequence that occurs at the boundary of an exon and an intron (splice site). This change can disrupt messenger RNA splicing resulting in the loss of exons or the inclusion of introns and an altered protein-coding sequence.

A frameshift mutation or variant a genetic mutation caused by indels (insertions or deletions) of a number of nucleotides in a DNA sequence that is not divisible by three. Due to the triplet nature of gene expression by codons, the insertion or deletion can change the reading frame (the grouping of the codons), resulting in a completely different translation from the original. The earlier in the sequence the deletion or insertion occurs, the more altered the protein. A frameshift mutation will in general cause the reading of the codons after the mutation to code for different amino acids. The frameshift mutation will also alter the first stop codon (“UAA”, “UGA” or “UAG”) encountered in the sequence. The polypeptide being created could be abnormally short or abnormally long, and will most likely not be functional.

A 5′ UTR mutation or variant refers to a mutation The 5′ untranslated region (5′ UTR) (also known as a leader sequence or leader RNA) is the region of an mRNA that is directly upstream from the initiation codon. This region is important for the regulation of translation of a transcript by differing mechanisms in viruses, prokaryotes and eukaryotes. The 5′ UTR has been found to interact with proteins relating to metabolism, and proteins translate sequences within the 5′ UTR. In addition, this region has been involved in transcription regulation. Regulatory elements within 5′ UTRs have also been linked to mRNA export.

A splice site mutation is a genetic mutation that inserts, deletes or changes a number of nucleotides in the specific site at which splicing takes place during the processing of precursor messenger RNA into mature messenger RNA. Splice site consensus sequences that drive exon recognition are located at the very termini of introns. The deletion of the splicing site results in one or more introns remaining in mature mRNA and may lead to the production of abnormal proteins. When a splice site mutation occurs, the mRNA transcript possesses information from these introns that normally should not be included. Introns are supposed to be removed, while the exons are expressed.

Nonsense (Stop-gain) is a mutation that results in a premature termination codon (a stop was gained), which signals the end of translation. This interruption causes the protein to be abnormally shortened. The number of amino acids lost mediates the impact on the protein's functionality and whether it will function whatsoever.

An in-frame deletion involves at least 3 DNA bases (it may be more and is usually multiple of 3) removes an entire codon and so may lead to the deletion of an amino acid from a protein e.g. in-frame deletion.

Further, the gene may be mutated due to a chromosomal translocation which is a type of chromosomal abnormality in which a chromosome breaks and a portion of it reattaches to a different chromosome or portion thereof.

In an additional embodiment, the present invention provides a method of increasing serum copper levels in a subject by administering copper histidinate at a dose of about 1450-8900 μg daily to the subject, wherein the subject is four weeks old or younger at initial administration, thereby increasing serum copper levels. In one aspect, the subject is four weeks old or younger, less than one year old or greater than one year old. In another aspect, the administration is once or twice a day. In an additional aspect, the copper histidinate is administered at about 1450 μg once or twice daily. In a further aspect, the administration is by subcutaneous injection. In one aspect, the subject has Menkes disease. In a further aspect, the subject has a mutation in the ATP7A gene. In various aspects, the mutation in the ATP7A gene is a missense variant, intron variant, synonymous variant, splice region variant, frameshift variant, splice acceptor variant, 5′ UTR variant, splice donor variant, nonsense (stop gained), duplicative variant, in frame deletion, gross deletion, chromosomal translocation or a combination thereof. In certain aspects, the serum copper, serum ceruloplasmin, serum bicarbonate, serum creatinine and/or urine beta-2 microglobulin levels of the subject are monitored. In various aspects, the serum copper level of the subject prior to administration of the copper histidinate is less than 75 μg/dl. In one aspect, following administration of copper histidinate to the subject, the serum copper level of the subject will be at least about 85 μg/dl.

In one aspect, the subject has normal levels of serum copper and the administration of copper histidinate increases the level of serum copper above normal level. In an additional aspect, the subject has low levels of serum copper and administration of copper histidinate increase the levels of serum copper to normal levels or above normal levels. In one aspect, a normal serum copper level is about 85-180 μg/dl. In an additional aspect, a subject with low levels of serum copper of <85 μg/dl. In a specific aspect, the subject has serum copper levels <75 μg/dl. In a further aspect, the subject has a disease or disorder that causes or results in low serum copper levels. In certain aspects, the disease or disorder is Menkes disease.

Following administration of copper histidinate, levels of the serum copper, serum ceruloplasmin, serum bicarbonate, serum creatinine and/or urine beta-2 microglobulin levels are monitored. In certain aspects, the levels of copper histidinate, levels of the serum copper, serum bicarbonate, serum ceruloplasmin, serum creatinine and/or urine beta-2 microglobulin levels are measured to determine the levels are higher or lower than normal levels. In one aspect, a normal serum copper level is about 85-180 μg/dl. In an additional aspect, a low level of serum copper is <85 μg/dl. In a specific aspect, the subject has serum copper levels <75 μg/dl. In some aspects, a normal level of serum bicarbonate is 22-29 mmol/L. In certain aspects, a low level of serum bicarbonate is <22 mmol/L and a high level of serum bicarbonate is >29 mmol/L. In one aspect, a low level of serum bicarbonate is <12 mmol/L. In one aspect, a normal level of serum creatinine is 0.2-0.4 mg/dl. In various aspects a low level of serum creatinine is <0.2 mg/dl and a high level of serum creatinine is >0.4 mg/dl. In some aspects, a high level of serum creatinine is >0.5 mg/dl. In certain aspects, a normal level of urine beta-2 microglobulin is about 0-0.3 mg/L. In various aspects, a normal level of serum ceruloplasmin is 20-35 mg/dl. In one aspect a low level of ceruloplasmin is <20 mg/dl. In certain aspects, a high level of serum ceruloplasmin is >30 mg/dl. In some aspects, a high level of urine beta-2 microglobulin is >3 mg/L. In one aspect, a high level of urine beta-2 microglobulin is >75 mg/dl.

In a further embodiment, the present invention provides a pharmaceutical composition of lyophilized copper histidinate and a pharmaceutically acceptable carrier. In one aspect, the pharmaceutically acceptable carrier is saline. In an additional aspect, the lyophilized copper histidinate is reconstituted with saline or other carrier. The saline can be buffered (e.g., phosphate buffered saline (PBS)). In a further aspect, the reconstituted copper histidinate has a concentration of about 500 μg/ml to 3000 μg/ml, for example 2900 μg/ml.

As used herein, the phrase “pharmaceutically acceptable carrier” includes any and all solvents, solutions, buffers, dispersion medias, coatings, antibacterial and antifungal agents, isotonic and absorption delaying agents, and the like, that are compatible with pharmaceutical administration. Such a composition may contain, in addition to the antibodies of the invention and carrier, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration. In one aspect, the pharmaceutically acceptable carrier is saline, e.g., 0.9% saline.

In yet a further embodiment, the present invention provides a kit with lyophilized copper histidinate; and directions for use. In one aspect, the lyophilized copper histidinate is reconstituted with saline. In an additional aspect, the lyophilized copper histidinate is reconstituted with 0.2-2 ml of saline. In a further aspect, the reconstituted copper histidinate is stored at about 2° C.-37° C. In one aspect, the lyophilized product is stored at about −10 to −20° C. In certain aspects, the kit contains directions or instructions for reconstituting the copper histidinate. In various aspects, the kit contains an apparatus to reconstitute the copper histidinate. In specific aspects, the apparatus to reconstitute the copper histidinate is a syringe, a vial or an ampoule with a hypodermic needle. In an additional aspect, the copper histidinate is reconstituted with 2.0 ml of saline. In a further aspect, the concentration of the reconstituted copper histidinate is equivalent to elemental copper concentration of about 100 to 3000 μg/ml. In various aspects, the concentration of the reconstituted copper histidinate is equivalent to elemental copper concentration of 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 950 μg/ml, 1000 μg/ml, 1500 μg/ml, 2000 μg/ml, 2500 μg/ml or 3000 μg/ml. In certain aspects, the concentration of the reconstituted copper histidinate is equivalent to element copper concertation of 500 μg/ml. In one aspect, the kit contains directions or instructions for administering the reconstituted copper histidinate to a subject. In various aspects, the subject is administered 0.5 ml of the reconstituted copper histidinate solution. In an additional aspect, the subject is administered 250 μg of the reconstituted copper histidinate solution. In a further aspect, the kit contains directions or instructions for storing the reconstituted copper histidinate solution.

In one embodiment, by way of example, the present invention provides a stable lyophilized pharmaceutical formulation of copper histidinate, wherein the formulation made by lyophilizing an aqueous solution comprising 2 moles of L-histidine and 1 mole of cupric chloride (for example, Copper II Chloride Dihydrate) and adjusted to a pH of about 7.35 in an aqueous solution, followed by lyophilization of the solution. In one aspect, the copper histidinate is present at about 2900 μg/mL in the aqueous solution. In an additional aspect, the formulation is stable for at least about 18 months. In one aspect, a formulation stable for 18 months is stored at about −10 to −20° C., 2 to 8° C., or room temperature (15 to 30° C.). In an additional embodiment, the present invention provides a vial comprising a formulation of copper histidinate and a pharmaceutically acceptable carrier, wherein the formulation is made by lyophilizing an aqueous solution comprising 2 moles of L-histidine and 1 mole of cupric chloride (for example, Copper II Chloride Dihydrate) and adjusted to a pH of about 7.35 in an aqueous solution, followed by lyophilization of the solution. In one aspect, the pharmaceutically acceptable carrier is saline. In an additional aspect, the formulation is reconstituted in 2 ml of saline to a concentration of about 2900 μg/ml. In a further aspect, the formulation comprises lyophilized copper histidinate reconstituted in saline at a concentration of 500 μg elemental copper/ml in the vial. In certain aspects, the vial further comprises instructions for reconstitution of the lyophilized copper histidinate in saline and subcutaneous administration to a subject with Menkes disease at a dose of 0.5 ml (or cc) per administration twice daily until the subject is one year of age and once daily after the subject is one year of age. In one aspect, the lyophilized copper histidinate is stable at about 2-8° C., e.g., 4° C. for at least about 7 days, 2 weeks, one month, or greater than 2 months. In certain aspects, the subject with Menkes has an ATP7A mutation selected from a nonsense variant, missense variant, intron variant, synonymous variant, splice region variant, frameshift variant, splice acceptor variant, 5′ UTR variant, splice donor variant, duplicative variant, in frame deletion, gross deletion, chromosomal translocation, canonical splice site mutation or a combination thereof.

In one aspect, a vial of the invention includes instructions for reconstitution of the lyophilized copper histidinate in saline and subcutaneous administration to a subject with Menkes disease at a dose of 0.5 ml per administration twice daily until the subject is one year of age and once daily after the subject is one year of age. In one aspect, the subject with Menkes has an ATP7A mutation selected from a nonsense variant, missense variant, intron variant, synonymous variant, splice region variant, frameshift variant, splice acceptor variant, 5′ UTR variant, splice donor variant, duplicative variant, in frame deletion, gross deletion, chromosomal translocation, canonical splice site mutation or a combination thereof. In another aspect, the instructions include performing a genetic test to identify an ATP7A mutation in the subject.

In yet another embodiment, the invention provides a container with two chambers for mixing a powdered medicament, in this instance lyophilized copper histidinate, with a diluent such as saline, prior to injecting the mixed ingredients into a subject through a needle externally affixed to the container. The container can be a syringe, a vial, an ampoule or the like.

The Tₘₐₓ (time of peak plasma concentration) is the time required to reach maximum drug concentration in the plasma after drug administration. Tₘₐₓ is peak plasma time. Simply, Tₘₐₓ is the time to reach Cₘₐₓ. Cₘₐₓ is the maximum (peak) plasma drug concentration attained after the oral administration of the drug. Also, at Tₘₐₓ, the drug absorption rate exactly equals the drug elimination rate.

On the other hand, t_{1/2} (elimination half-life) is the time required to decrease the drug concentration within the body by one-half during elimination. In other words, t_{1/2} (elimination half-life) is the time required for the amount or concentration of a drug to decrease by one-half. In pharmacokinetic, elimination half-life, t_{1/2} refers to the time required for half the dose of drug administered to be removed from the body. The Tₘₐₓ is dependent on the elimination rate (k) and absorption rate constant (ka). while t_{1/2} is dependent on the elimination rate (k) and volume of distribution (Vd). At Tₘₐₓ, the concentration of a drug is in peak. While at t_{1/2}, the concentration of a drug may or may not in peak. See for example, https://pharmaeducation.net/difference-between-tmax-and-t1-2/#:˜:text=On %20the %20other %20hand %2C %20t%201%2F2%20%28elimination%20half- life%29,of%20a%20drug%20to%20decrease%20by%20one-half%20%5B1%5D.

In an illustrative example herein, the disclosure provides a method of increasing serum copper histidinate levels in a subject by administering copper histidinate to the subject via a subcutaneous route of administration with a peak detectable level of copper histidinate in the serum at about ½ hour to 1 hour following administration, thereby increasing serum copper histidinate levels in the subject. Peak detectable levels of copper histidinate can be from ¼ hour to 2 hours following administration. Further copper histidinate distribution phase half-life is about 1-5 hours, about 1-4 hours, about 1-3 hours, about 2-5 hours, about 2-4 hours, or about 2-3 hours.

In one aspect, the terminal elimination phase half-life t½ is at about 30-150 hours. In other aspects the terminal elimination phase half-life t½ is at about 30-125 hours, about 30-100 hours, about 30-75 hours, or about 75 hours. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Copper Histidinate Treatment of Menkes Patients

The purpose of this study was to allow currently enrolled participants to complete a three-year course of subcutaneous copper histidinate treatment under a previous protocol. It was hypothesized that subcutaneous injections of this drug will raise serum copper levels and ceruloplasmin levels in enrolled participants, improve neurodevelopmental and neurological outcomes, and reduce mortality compared to untreated affected subjects. The broad overall hypothesis of this treatment study is that subcutaneous copper histidinate injections will normalize circulating copper levels and provide health benefits to individuals with copper deficiency due to genetic abnormalities such as classic Menkes disease or which is unexplained. The trial represents an open-label design in which all enrolled subjects receive subcutaneous injections of copper histidinate. Immediate potential benefits of Copper Histidinate treatment in classic Menkes disease include protection against seizure activity and accrual of neurodevelopmental skills in an age-appropriate fashion. Long-term potential benefits include enhanced lifespan and quality of life, as noted in other subjects with classic Menkes disease treated with Copper Histidinate.

Copper histidinate, the active substance, is a 1:2 complex of Copper (II) with L-histidine formed in situ when 1 mole of Cupric Chloride is reacted with 2 moles of L-histidine in aqueous solution and adjusted to approximately pH 7.35. The IUPAC chemical name is (2S)-2-amino-3-(1H-imidazol-5-yl) propanoic acid, Copper. The molecular formula is C₁₂H₁₈CuN₆O₄ (molecular weight: 373.86 g/mol). In a particular embodiment, the invention provides a study intervention with administration of copper histidinate in dose(s) prescribed as follows: 1450 μg subcutaneous twice a day (BID) in infants up to 12 months of age, and 1450 μg subcutaneous once a day (QD) for infants and children older than 12 months. In one illustrative example, the total duration of copper histidinate treatment does not exceed three years. Copper histidinate is administered by subcutaneous injection in a volume not to exceed 0.5 ml using an syringe with a #27 gauge needle, by way of example. The standard dosing regimen used for classic Menkes disease is copper histidinate 1450 μg (0.5 ml) subcutaneous BID for patients younger than 12 months of age, and 1450 μg subcutaneous QD for patients older than 12 months. This regimen typically maintains serum copper levels in the normal range for such subjects.

Inclusion criteria for patients included being diagnosed with classic Menkes disease, Occipital Horn Syndrome (OHS), or unexplained copper deficiency; Serum copper level results between 0 and 75 mg/dl (normal range 80-180 μg/dl); and ability to adhere to the prescribed subcutaneous Copper Histidinate injection regimen.

Exclusion criteria for patients included pre-existing liver (e.g., hepatitis, biliary atresia, cirrhosis) or kidney disease (e.g., serum creatinine >1.0 mg/dL); history of bleeding diatheses; pregnancy or lactation; diagnosis of Wilson disease; any disease or condition that has a high probability of precluding the patient from completing the study or where the patient cannot or will not appropriately comply with study requirements; participation in any other investigational trial in which receipt of investigational drug or device occurred within 30 days prior to screening for this study; history of diagnosed drug or alcohol dependence within the previous 3 years; any disease process that may adversely affect gastrointestinal absorption, e.g. celiac sprue; chronic/severe cardiac disease (applies to adult subjects only) that could make participating in a clinical trial physically demanding, including but not limited to cardiac insufficiency, arrhythmias, bradycardia, or hypotension, unless associated with other features of dysautonomia, as in OHS; and history of cerebrovascular accident (applies to adult subjects only) that could make participating in a clinical trial difficult for the subject.

Serum copper levels were tracked to allow a decision as to whether the copper levels are abnormally high, and to adjust the intervention accordingly. In addition, serum bicarbonate, serum creatinine, and urine β-2-microglobulin (markers of renal tubular function) were also tracked (Table 1) and used to institute changes in copper histidinate dose if necessary.

TABLE 1

| | Reference | Dose |
|---|---|---|
| Clinical Test | Ranges | Adjustment |
| Serum copper >175 μg/dl | 80-180 | Stop CuHis for 7 days; repeat test and |
| | μg/dL | resume dose on every other day schedule |
| | | (QOD) if ≤125 μg/dL; re-measure after 4 |
| | | weeks. |
| Serum Bicarbonate <12 mmol/L | 22-29 | Repeat measurement; If confirmed, |
| | mmol/L | stop CuHis for 7 days; resume dose on |
| | | QOD schedule. Re-measure after 4 |
| | | weeks. |
| Serum Creatinine >0.5 mg/dL | 0.2-0.4 | Repeat measurement; If confirmed, |
| | mg/dL | stop CuHis for 7 days; resume dose on |
| | | QOD schedule. Re-measure after 4 |
| | | weeks |
| Urine beta-2-microglobulin >75 | 0-0.3 | Correlate with serum bicarbonate and |
| mg/L | mg/L | creatinine level |

The immediate risks of renal copper overload involve acid-base imbalance due to increased loss of bicarbonate. This will be addressed by stopping copper histidinate administration for one week and lowering the dose for an additional three weeks before re-measuring the laboratory values. The potential long-range risk would be permanent kidney damage requiring dialysis or renal transplantation. However, there is evidence that renal tubular injury in classic Menkes disease patients caused by copper histidinate is reversible.

At the end of this study patients were transferred to a long-term study to determine survival, neurological development and quality of life for the patients.

### Example 2

### Long Term Study of Copper Histidinate Treatment for Menkes Disease

This study investigated the clinical effects and safety of subcutaneous copper histidinate treatment for patients diagnosed with classic Menkes disease and newly diagnosed Menkes disease patients.

During the study patients were administered does of copper histidinate as follows: 1450 μg subcutaneous BID in infants up to 12 months of age, and 1450 μg subcutaneous QD for infants and children older than 12 months. In one example, the total duration of copper histidinate treatment for newly diagnosed Menkes disease patients did not exceed three years.

Previously diagnosed patients had a confirmed diagnosis based on clinical, biochemical and molecular criteria. The clinical criteria included abnormal hair color and/or texture, and/or seizures, and/or hypotonia, and/or developmental delay. Biochemical criteria included low serum copper levels (<75 μg/dl) and/or ceruloplasmin and/or abnormal plasma catecholamine levels. Molecular criteria included mutations in the ATP7A gene (deletion/duplication, non-sense, missense or canonical or non-canonical splice junction mutations). Newly diagnosed patients had serum copper levels <75 μg/dl and were tested for ATP7A gene mutation(s).

Any patient with pre-existing liver (e.g., hepatitis, biliary atresia, cirrhosis) or kidney disease (e.g., serum creatinine >1.0 mg/dl); history of bleeding diatheses; or diagnosis of Wilson disease was excluded from the study.

Newly diagnosed patients were monitored for copper toxicity by laboratory analyses to include: basic metabolic panel (BMP), serum copper, ceruloplasmin and urine beta-2 microglobulin concentrations every 6 weeks for the first 6 months. The patients were evaluated in clinic monthly for the first 3-4 months then every 2-3 months thereafter. Patients were assessed to determine survival status approximately every 3 months by either telephone contact and/or visit. The BMP panel includes albumin, alkaline phosphatase, ALT, AST, bicarbonate, bilirubin (total and direct), BUN, calcium, chloride, creatinine, creatinine kinase, glucose, LDH, magnesium, phosphorous, potassium, total protein, and sodium.

Previously diagnosed patients were monitored for copper toxicity by laboratory analyses to include: BMP, serum copper, ceruloplasmin and urine beta-2 microglubulin concentrations initially when initially enrolled and then every 3-4 months until completing 3 years of treatment. Patients were assessed to determine survival status approximately every 3 months by either telephone contact and/or visit.

Any changes to the dose of copper histidinate was determined by the safety parameters in Table 2.

TABLE 2

| | Reference | Dose |
|---|---|---|
| Clinical Test | Ranges | Adjustment |
| Serum copper >175 μg/dl | 80-180 | Stop CuHis for 7 days; repeat test |
| | μg/dL | andresume dose on QOD schedule |
| | | if ≤125 μg/dL; Re-measure |
| | | after 4 weeks. |
| Serum Bicarbonate <12 mmol/L | 22-29 | Repeat measurement; If confirmed, |
| | mmol/L | stop CuHis for 7 days; resume dose |
| | | on QOD schedule. Re-measure after |
| | | 4 weeks. |
| Serum Creatinine >0.5 mg/dL | 0.2-0.4 | Repeat measurement; If confirmed, |
| | mg/dL | stop CuHis for 7 days; resume dose |
| | | on QOD schedule. Re-measure after |
| | | 4 weeks |
| Urine beta-2-microglobulin >75 | 0-0.3 | Correlate with serum bicarbonate |
| mg/L | mg/L | and creatinine level |

### Example 3

### Long Term Follow Up of Menkes Disease Patients Treated with Copper Histidinate

This study was designed as a long term follow up on patients previously identified and/or enrolled in previous studies for the treatment of Menkes disease with copper histidinate and untreated patients to determine survival, neurological development, quality of life status and adverse events. This study followed the patients from the previous examples for long term follow up.

Patients previously enrolled in clinical trials for the treatment of Menkes disease with copper histidinate were assessed for survival status, neurological development status and quality of life. No drug was be administered during this study.

The results indicated that Menkes disease subjects who received early treatment with copper histidinate had a nearly 80% reduction in the risk of death (hazard ratio=0.21, p<0.00001) compared with untreated historical controls. The median survival or the early treatment cohort was 14.8 years (177.1 months compared to 1.3 years (15.9 months) for the untreated historical control group.

### Example 4

### Pharmacokinetics Study

This study is a phase I, open label, single dose study to evaluate the pharmacokinetics, safety and tolerability of copper histidinate after subcutaneous administration in healthy male volunteers.

In previous studies with Menkes patients, children less than 1 year of age received 1450 μg copper histidinate (equivalent to 250 μg elemental copper) twice daily, while children older than 1 year of age received 1450 μg copper histidinate (equivalent to 250 μg elemental copper) once daily. This dose has been chosen to target adequate serum copper exposure in Menkes children.

Menkes children on copper replacement therapy are expected to weigh at or less than the lowest 3rd percentile of normal children. Because the safety of subcutaneous copper histidinate in healthy volunteers without copper deficiency is unknown, the lower weight-adjusted dose from the 3-year old Menkes child is targeted. A 3-year old male at the 3rd percentile for weight is expected to weigh 11.8 kg, while a typical man weighs 70 kg.

Using allometric scaling with an exponent of 0.75, the equivalent adult dose is about 5800 μg copper histidinate (equivalent to 1000 μg of elemental copper, which also happens to be maximum amount of copper that can be absorbed through the intestines under normal condition. Therefore, a single subcutaneous dose of 5800 μg copper histidinate (equivalent to 1000 elemental copper) will be studied. Assuming an allometric exponent of 1, the equivalent adult dose is 8850 μg copper histidinate (equivalent to 1500 μg elemental copper). Therefore, 8850 copper histidinate (equivalent to 1500 μg elemental copper) will be the maximum dose considered in this study.

The study consists of a 28-day screening phase, an approximately 96-hour stay at the clinical research unit (CRU), and a follow up phone call 4 (±1) days after CRU discharge. A total of 9 healthy males will receive a single dose of 5800 μg copper histidinate (equivalent to 1000 μg of elemental copper) administered subcutaneous, in 2 separate injections, each in a different arm on Day 2. Each 1 mL injection will contain 2900 μg of copper histidinate (equivalent to 500 μg of elemental copper).

Subjects will be screened within 28 days (Days −29 to −2) prior to admission to the CRU. Subjects will be admitted to the CRU late afternoon of Day −1 with dinner provided, followed by an overnight fast. Subjects will follow a standardized meal schedule with known and consistent copper contents as well as adhere to a fixed sleep-wake schedule beginning on Day −1 and lasting until CRU discharge on Day 4. The start of the first subcutaneous copper histidinate injection will be defined as time 0 on Day 2.

Baseline assessments will commence on Day 1 and will include blood sampling corresponding to the Day 2 schedule to assess the baseline diurnal variation in serum copper, ceruloplasmin, and copper histidinate levels. Blood for pharmacokinetic analysis for Day 1 will be collected at 1 hour after waking up at approximately 7 AM, then at 0.25, 0.5, 0.75, 1, 1.5, 2, 3, 4, 6, 8, 10, 12, 14, 16, and 24 hours (pre-dose Day 2) after the first Day 1 pharmacokinetic sample. On Day 2, a sample will be collected within 15 minutes prior to subcutaneous copper histidinate injection (in a different tube from the one used for the 24-hours post first Day 1 pharmacokinetic sample), then at 0.25, 0.5, 0.75, 1, 1.5, 2, 3, 4, 6, 8, 10, 12, 14, 16, 24, 28, 32, 36, and 48 hours after subcutaneous copper histidinate injection. Time 0 hours on Day 2 will correspond approximately to 8 AM; dosing should be initiated as soon as possible (within 15 minutes) after collecting the pre-dose sample.

Urine samples will be collected for pharmacokinetic analysis starting on Day 1 through Day 4 in defined time intervals. Urine will be collected from 0 to 6, 6 to 12, and 12 to 24 hours after the first pharmacokinetic blood sample of Day 1 (before subcutaneous copper histidinate injection). After subcutaneous copper histidinate injection, urine will be collected from 0 to 6, 6 to 12, 12 to 24, and 24 to 48 hours after drug administration.

Safety parameters, including serious adverse events/adverse events (AEs/SAEs), resting vital signs, ECG, laboratory evaluations, physical examinations and concomitant medications will be collected throughout the study. AEs will be simply reported at any time throughout the study when they occur. Before subcutaneous injection of copper histidinate and at each blood sample collection, subjects will be assessed for injection site reactions. Vital signs will be measured at Screening, before baseline assessment on Day 1, pre-dose on Day 2, and at 0.5, 1, 2, 4, 12, 24, and 48 hours after subcutaneous copper histidinate injection. ECGs will be measured at Screening, pre-dose on Day 2, and 1, 2, 4, and 24 hours post-dose. Clinical laboratory panels will be taken at Screening, upon CRU check-in, and 24 (Day 3) and 48 (Day 4) hours after subcutaneous copper histidinate dosing. Full physical examination will be performed at Screening, upon CRU check-in, and 24 (Day 3) and 48 (Day 4) hours after subcutaneous copper histidinate dosing, with a brief physical examination conducted at 2 hours after dosing on Day 2.

Copper histidinate, the active substance, has an approximate pH of 7.0-7.5, for example, 7.35, and belongs to the class of drugs for copper replacement therapy. The molecular formula is C₁₂H₁₈CuN₆O₄ with a molecular weight of 373.86 g/mol. The drug product, copper histidinate for subcutaneous injection, is lyophilized The lyophilized powder product is reconstituted with normal saline for subcutaneous injections. Two vials of copper histidinate lyophilized powder will be used for each subject. Prior to dosing, each 5 mL vial is reconstituted with 2 mL of 0.9% saline for injection.

A single dose of 5800 μg copper histidinate (equivalent to 1000 μg of elemental copper) will be administered via 2 subcutaneous injections in different arms. Two vials of drug product will each be reconstituted with 2 mL of 0.9% saline. From each vial, 1 mL of the reconstituted drug product will then be administered as separate subcutaneous injections for a total of 2 injections. The 2 injections should be given within about 5 minutes, in different arms for each subject.

Additional single doses up to 8850 μg copper histidinate (equivalent to 1500 μg elemental copper) may be administered in an additional cohort via 2 subcutaneous injections in different arms. Two vials of drug product will each be reconstituted with 2 mL of 0.9% saline. From each vial, 1.5 mL of the reconstituted drug product will then be administered as separate subcutaneous injections for a total of 2 injections. The 2 injections should be given within 5 minutes, in different arms, for each subject.

Pharmacokinetic assessments: Blood samples for pharmacokinetic analysis of serum copper, ceruloplasmin, and copper histidinate will be collected on Day 1 at 1 hour after waking (time 0 hours of Day 1), and at 0.25, 0.5, 0.75, 1, 1.5, 2, 3, 4, 6, 8, 10, 12, 14, 16, and 24 hours (pre-dose Day 2) after the first Day 1 pharmacokinetic sample. On Day 2, a sample will be collected within 15 minutes prior to subcutaneous copper histidinate injection (in a different tube from the one used for the 24-hours post first Day 1 pharmacokinetic sample), and at 0.25, 0.5, 0.75, 1, 1.5, 2, 3, 4, 6, 8, 10, 12, 14, 16, 24, 28, 32, 36, and 48 hours after subcutaneous copper histidinate injection.

Urine samples will be collected for pharmacokinetic analysis of copper starting on Day 1 through Day 4 in defined time intervals. Subjects should empty their bladders (sample can be discarded) approximately 30 minutes prior to the initiation of baseline assessment at time 0 hours on Day 1. Urine will be collected from 0 to 6, 6 to 12, and 12 to 24 hours after time 0 hours (before subcutaneous copper histidinate injection). After subcutaneous copper histidinate injection, urine will be collected from 0 to 6, 6 to 12, 12 to 24, and 24 to 48 hours after drug administration.

Full physical examinations will be performed at Screening, upon CRU check-in, and 24 (Day 3) and 48 (Day 4) hours after subcutaneous copper histidinate dosing, with a brief physical examination conducted at 2 hours after dosing on Day 2.

Pharmacokinetic parameters for diurnal variation-adjusted serum copper, ceruloplasmin, and serum copper histidinate after single doses of subcutaneous copper histidinate, derived from observed serum and urine concentration data using standard noncompartmental analysis (NCA) methods, will include the following: Peak concentration (Cₘₐₓ); Time to peak concentration (Tₘₐₓ); Area under the concentration-time curve (AUC) from time zero to last quantifiable concentration (AUCₗₐₛₜ); AUC from time zero to infinity (AUC_{inf}); Apparent total body clearance (CL); Apparent renal clearance (CLr); Elimination half-life (t_{1/2}); Cumulative amount (in μg and as a percentage of the dose) of copper excreted into the urine (Ae) from time zero to 6 hours (Ae₀₋₆), 14 hours (Ae₀₋₁₄), 24 hours (Ae₀₋₂₄), and 48 h (Ae₀₋₄₈) after subcutaneous injection, and from time zero to infinity (Ae_{inf}).

Predicted peak and total exposure (Cₘₐₓ and AUCₜₐᵤ) of diurnal variation-adjusted serum copper, ceruloplasmin, and copper histidinate at steady state dosing will be also analyzed.

### Example 5

### Pharmacokinetic Studies in Adult Humans

The following Example describes the pharmacokinetics of copper histidinate as determined by a study that administered 3000 μg (3.0 mg) (˜500 nCi) [¹⁴C]-CuHis solution subcutaneous injection to six healthy adult male volunteers once daily.

The primary objective of this study was to quantitate total radioactivity (TRA) in whole blood, serum, and the eluted fractions of serum spiked with unlabeled copper histidinate (CuHis) from a size exclusion-high performance liquid chromatography (SEC-HPLC) column after a single subcutaneous (SC) administration of [¹⁴C] CuHis in healthy adult male subjects. Further, the study was designed to determine the percentage of ¹⁴C radioactivity associated with cellular components in whole blood over time (e.g., whole blood:serum partitioning concentration ratio).

To evaluate the safety and tolerability after a single SC administration of [¹⁴C] CuHis in healthy adult male subjects. This was an open-label, single-dose, 1-period, PK study conducted at 1 study center in the USA. Six (6) healthy, adult male subjects were enrolled. Screening of subjects occurred within 28 days prior to dosing.

On Day 1, a single SC 3.0 mg (˜500 nCi) dose of [¹⁴C]-CuHis was administered. Pharmacokinetic (PK) samples for determining TRA in whole blood and serum, and [¹⁴C] CuHis in eluted fractions of serum spiked with unlabeled CuHis from an SEC-HPLC column were collected pre-dose and up to 168 hours post-dose (Subjects 1 to 5) or 504 hours post-dose (Subject 6).

Subjects were housed on Day −1, at the time indicated by the clinical research unit (CRU), until after the 168-hour blood draw and/or study procedures, and returned for the 504-hour blood draw if possible. Safety was monitored throughout the study by repeated clinical and laboratory evaluations. The CRU attempted to contact all subjects who received the study drug using their standard procedures approximately 14 days after dosing to determine if any adverse event (AE) had occurred since the last study visit.

The initial pharmacokinetic results for ¹⁴C copper histidinate were as follows:
AUC₀₋₂₄ (ng·hr/mL)=185.6 (20.6)*
AUC₀₋ₗₐₛₜ (ng·hr/mL)=249.3 (10.6)*
AUC_{0-∞} (ng·hr/mL)=296.0 (15.3)*
Cₘₐₓ (ng/mL)=67.14 (35.9)*
Tₘₐₓ (hr)=0.747 (0.50, 0.76)** *AUC₀₋₂₄, AUC₀₋ₗₐₛₜ, AUC_{0-∞}, and Cₘₐₓ values are presented as geometric mean (geometric percent coefficient of variation).**Tₘₐₓ values is presented as median (minimum, maximum).
   After peaking at 0.75 hours post-dose, serum [¹⁴C]-CuHis concentrations exhibited an initial rapid decrease (distribution phase; t ½=˜2-3 hours), followed by a slower equilibrium and/or terminal elimination phase (t ½=˜75 hours).

A single dose of 3.0 mg (˜500 nCi) [¹⁴C]-CuHis (equivalent to approximately 500 μg of elemental copper) for SC bolus injection was administered into the upper arm of each subject at Hour 0 on Day 1. Hour 0 corresponded to the start of the bolus SC administration on Day 1. Subjects were instructed not to rub the injection site(s). The actual doses of CuHis and radioactivity administered to each subject is presented in TABLE 3 below.

TABLE 3
Actual Doses of Copper Histidinate and Radioactivity Administered
(Target Dose: 3.0 mg (~500 nCi) [¹⁴C]-Copper
Histidinate) (Safety Population)

| | Actual Doses Administered | |
|---|---|---|
| Subject Number | Copper Histidinate (mg) | Radioactivity (nCi) |
| 1 | 3.13 | 493 |
| 2 | 3.07 | 485 |
| 3 | 2.99 | 471 |
| 4 | 3.13 | 494 |
| 5 | 3.03 | 479 |
| 6 | 2.92 | 478 |
| n | 6 | 6 |
| Mean | 3.045 | 483.4 |
| SD | 0.081341 | 9.0103 |
| CV % | 2.7 | 1.9 |
| SEM | 0.033207 | 3.6784 |
| Minimum | 2.92 | 471 |
| Median | 3.053 | 481.9 |
| Maximum | 3.13 | 494 |

Pharmacokinetics:

Blood samples for determining TRA in whole blood and serum, and [¹⁴C]-CuHis in eluted fractions of serum spiked with unlabeled CuHis from a SEC-HPLC column were collected predose and up to 168 hours postdose (Subjects 1 to 5) or up to 504 hours postdose (Subject 6).

TRA CuHis concentration equivalents in whole blood and serum were determined using accelerator mass spectrometry (AMS) at Pharmaron ABS, Inc. (Germantown, Maryland, USA). The lower limits of quantitation (LLOQ) for TRA CuHis concentration equivalents in whole blood and serum ranged from 0.711 to 0.770 ng eq CuHis/mL and 0.239 to 0.284 ng eq CuHis/mL in the different subjects, respectively.

[¹⁴C]-CuHis concentrations in serum fractions from the SEC-HPLC column were determined using AMS at Pharmaron ABS, Inc. (Germantown, Maryland, USA). The LLOQ for [¹⁴C]-CuHis concentrations in serum was 0.270 ng/mL.

Whole blood: serum TRA partitioning ratios were calculated at each postdose time point for each subject as the ratio between whole blood TRA CuHis concentration equivalents and serum TRA CuHis concentration equivalents. The following parameters were calculated for whole blood and serum TRA, and serum [¹⁴C]-CuHis, when possible: AUC₀₋₂₄, AUC₀₋ₗₐₛₜ, AUC_{0-∞}, AUC_{% extrap}, Cₘₐₓ, tₘₐₓ, t_{lag}, Kₑₗ, and t_{1/2}. The AUC₀₋₂₄ Ratio was calculated for serum [¹⁴C]-CuHis versus serum TRA. CL/F and V_{z}/F were calculated for serum [¹⁴C]-CuHis.

Statistical Methods:

All PK concentration and PK parameter descriptive statistics were generated using SAS® Version 9.4.

Whole blood and serum TRA CuHis concentration equivalents, serum [¹⁴C]-CuHis concentrations, and whole blood:serum TRA partitioning ratios were listed and summarized by time point for all subjects in the PK population. Summary statistics, including sample size (n), arithmetic mean (Mean), standard deviation (SD), coefficient of variation (CV %), standard error of the mean (SEM), minimum, median, and maximum were calculated for the above data at each nominal time point.

Whole blood and serum TRA PK parameters, and serum [¹⁴C]-CuHis PK parameters were listed for each subject and summarized for all subjects in the PK population. Summary statistics (n, Mean, SD, CV %, SEM, minimum, median, maximum, geometric mean [Geom Mean], and geometric CV % [Geom CV %]) were calculated for all PK parameters.

Profiles of arithmetic mean and individual whole blood and serum TRA CuHis concentration equivalents versus time and serum [¹⁴C]-CuHis concentrations versus time were presented on linear and semi-log scales. Profiles of arithmetic mean and individual whole blood:serum TRA partitioning ratios versus time were presented on linear scale. Linear mean plots were presented with and without SD.

Pharmacokinetic Results

Whole Blood and Serum Total Radioactivity Copper Histidinate Concentration Equivalents, Serum [¹⁴C]-Copper Histidinate Concentrations, and Whole Blood: Serum Total Radioactivity Partitioning Ratios

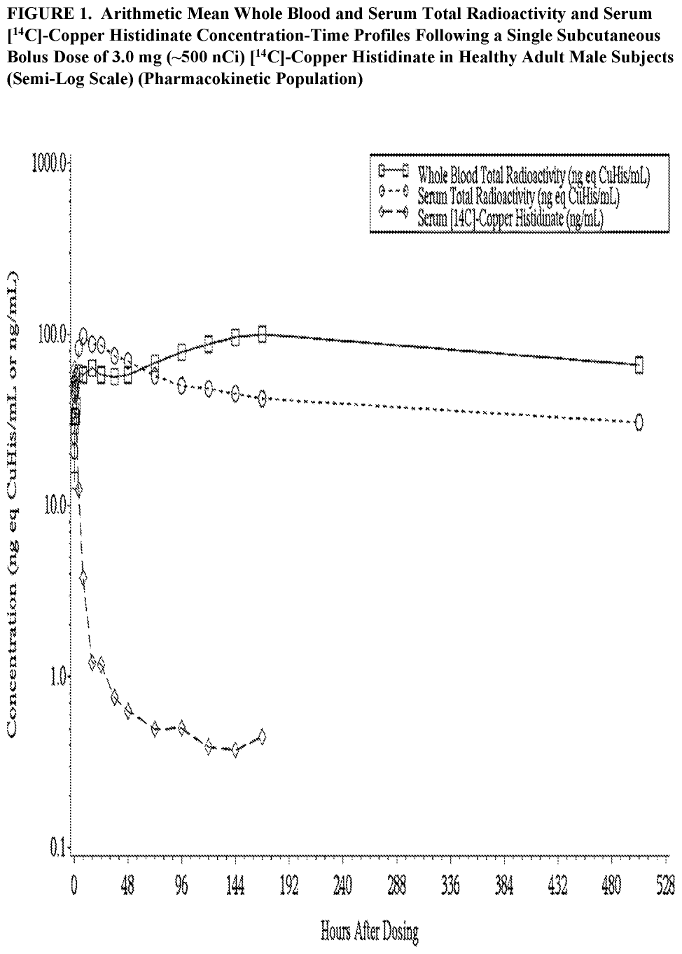

Arithmetic mean whole blood and serum TRA CuHis concentration equivalent versus time and serum [¹⁴C]-CuHis concentration versus time profiles following a single SC bolus dose of 3.0 mg (˜500 nCi) [¹⁴C]-CuHis in healthy adult male subjects are presented on semi-log scale (FIG. 1).

Arithmetic mean whole blood and serum TRA CuHis concentration equivalent versus time and serum [¹⁴C]-CuHis concentration versus time profiles following a single SC bolus dose of 3.0 mg (˜500 nCi) [¹⁴C]-CuHis in healthy adult male subjects are presented on semi-log scale (FIG. 1).

The data are exemplified in FIG. 1 which shows the arithmetic mean whole blood and serum total radioactivity and serum [¹⁴C]-Copper Histidinate concentration-time profiles following a single subcutaneous bolus dose of 3.0 mg (˜500 nCi) [¹⁴C]-Copper Histidinate in healthy adult male subjects (semi-log scale) (pharmacokinetic population).

Whole blood TRA CuHis concentration equivalents were quantifiable in all subjects by 0.25 hours postdose (i.e., the first scheduled postdose sample) and remained quantifiable in all subjects until their last sampling time point, i.e., 168 hours postdose for Subjects 1 to 5 and 504 hours postdose for Subject 6. Arithmetic mean whole blood TRA CuHis concentration equivalents rose up to 4 hours postdose where they remained in the 56.33 to 63.90 ng eq CuHis/mL range until 48 hours postdose, before rising again from 72 hours postdose onwards to its highest level of 100.9 ng eq CuHis/mL at 168 hours postdose. The whole blood TRA CuHis concentration equivalent of 66.3 ng eq CuHis/mL at 504 hours postdose is that obtained for Subject 6.

Serum TRA CuHis concentration equivalents were quantifiable in all subjects by 0.25 hours postdose and remained quantifiable in all subjects until their last sampling time point, i.e., 168 hours postdose for Subjects 1 to 5 and 504 hours postdose for Subject 6. The arithmetic mean serum TRA CuHis concentration equivalent-time profile peaked at 97.43 ng eq CuHis/mL at 8 hours postdose after which it declined in a multiphasic fashion. The serum TRA CuHis concentration equivalent of 30.7 ng eq CuHis/mL at 504 hours postdose is that obtained for Subject 6.

Serum [¹⁴C]-CuHis concentrations were quantifiable in all subjects by 0.25 hours postdose and remained quantifiable in all subjects until 24 hours postdose, and in at least half of the subjects until 168 hours postdose. The arithmetic mean serum CuHis concentration-time profile peaked at 63.47 ng/mL at 0.75 hours postdose after which they declined in a multiphasic fashion. As displayed, the multiphasic decline consisted of an initial rapid decrease (distribution phase) with a half-life estimated at approximately 2 to 3 hours followed by a slower apparent terminal elimination phase with a t_{1/2} of approximately 75 hours.

The arithmetic mean whole blood: serum TRA partitioning ratio versus time profile following a single SC bolus dose of 3.0 mg (˜500 nCi) [¹⁴C]-CuHis in healthy adult male subjects is presented on linear scale in the figure below, and the arithmetic mean (SD) ratios at each postdose time point are presented in TABLE 4 below.

TABLE 4
Summary of Whole Blood:Serum Total Radioactivity Partitioning
Ratios Following a Single Subcutaneous Bolus Dose of 3.0 mg
(~500 nCi) [¹⁴C]-Copper Histidinate in Healthy
Adult Male Subjects (Pharmacokinetic Population).

| | Whole Blood:Serum Total |
|---|---|
| Sampling Time | Radioactivity Partitioning Ratio |
| (Hour Postdose) | (Arithmetic Mean ± SD) (n = 6) |
| 0.25 | 0.6165 ± 0.10719 |
| 0.5 | 0.6410 ± 0.058110 |
| 0.75 | 0.6426 ± 0.059663 |
| 1 | 0.6888 ± 0.061072 |
| 1.5 | 0.6728 ± 0.054204 |
| 2 | 0.6761 ± 0.034459 |
| 4 | 0.7010 ± 0.093982 |
| 8 | 0.5856 ± 0.11748 |
| 16 | 0.7144 ± 0.11341 |
| 24 | 0.6708 ± 0.084839 |
| 36 | 0.7498 ± 0.048780 |
| 48 | 0.8329 ± 0.098113 |
| 72 | 1.209 ± 0.19245 |
| 96 | 1.612 ± 0.34610 |
| 120 | 1.853 ± 0.35117 |
| 144 | 2.163 ± 0.33332 |
| 168 | 2.392 ± 0.22198 |
| 504 | 2.160* |

*n = 1

TRA CuHis concentration equivalents were quantifiable in both whole blood and serum at all postdose sample times taken for all subjects, i.e., from 0.25 to 168 hour postdose, and arithmetic mean whole blood:serum TRA partitioning ratios ranged from 0.5856 to 2.392. Partitioning ratios were in the 0.5856 to 0.7498 range from 0.25 to 36 hours postdose and then increased from 0.7498 at 36 hours postdose to 2.392 at 168 hours postdose. Samples were only collected for Subject 6 at 504 hours postdose. The partitioning ratio at this time point was 2.16 for Subject 6.

Pharmacokinetic Parameters of Whole Blood and Serum Total Radioactivity, and Serum WO-Copper Histidinate

A summary of the PK parameters of whole blood and serum TRA and serum [¹⁴C]-CuHis following a single SC bolus dose of 3.0 mg (˜500 nCi) [¹⁴C]-CuHis in healthy adult male subjects is presented in TABLE 5 below.

TABLE 5
Summary of the Pharmacokinetic Parameters of Whole Blood and Serum Total Radioactivity and
Serum [¹⁴C]-Copper Histidinate Following a Single Subcutaneous Bolus Dose of 3.0 mg (~500
nCi) [¹⁴C]-Copper Histidinate in Healthy Adult Male Subjects (Pharmacokinetic Population)

| Pharmacokinetic | Whole Blood | Serum | Serum |
|---|---|---|---|
| Parameters | Total Radioactivity | Total Radioactivity | [¹⁴C]-Copper Histidinate |
| AUC_{0 24} (ng eq CuHis · hr/mL ^{a} | 1315 (30.4) [n = 6] | 1993 (21.4) [n = 6] | 185.6 (20.6) [n = 6] |
| or ng · hr/mL ^{b}) | | | |
| AUC₀₋ₗₐₛₜ (ng eq CuHis · hr/mL ^{a} | 12130 (15.3) [n = 5]^{c} | 9390 (13.4) [n = 5]^{c} | 249.3 (10.6) [n = 6] |
| or ng · hr/mL ^{b}) | | | |
| AUC_{0-∞} (ng eq CuHis · hr/mL ^{a} or | . | 23390 (25.7) [n = 5]^{c} | 296.0 (15.3) [n = 5]^{e} |
| ng · hr/mL ^{b})^{d} | | | |
| AUC_{% extrap} (%)^{d} | . | 58.34 ± 12.677 [n = 5]^{c} | 12.37 ± 8.3964 [n = 5]^{e} |
| AUC₀₋₂₄ Ratio | . | 0.09317 (19.5) [n = 6] | |
| Cₘₐₓ (ng eq CuHis/mL ^{a} or | 100.1 (14.8) [n = 6] | 95.78 (24.3) [n = 6] | 67.14 (35.9) [n = 6] |
| ng/mL ^{b}) | | | |
| tₘₐₓ (hr) | 168.006 (144.01, 168.03) [n = 6] | 8.007 (7.99, 16.02) [n = 6] | 0.747 (0.50, 0.76) [n = 6] |
| t_{lag} (hr) | 0.00 (0.00, 0.00) [n = 6] | 0.00 (0.00, 0.00) [n = 6] | 0.00 (0.00, 0.00) [n = 6] |
| Kₑₗ (1/hr)^{d} | . | 0.003278 ± 0.0015876 [n = 5]^{c} | 0.01660 ± 0.015118 [n =5]^{e} |
| t_{1/2} (hr)^{d} | . | 252.682 ± 113.8692 [n = 5]^{c} | 74.842 ± 49.1558 [n = 5]^{e} |
| CL/F (L/hr) | . | . | 10.34 ± 1.6214 [n = 5]^{e} |
| V_{z}/F (L) | . | . | 1034 ± 587.82 [n = 5]^{e} |

AUC₀₋₂₄, AUC₀₋ₗₐₛₜ, AUC_{0-∞}, AUC₀₋₂₄ Ratio, and Cₘₐₓ values are presented as geometric mean (geometric percent coefficient of variation).
AUC_{% extrap}, Kₑₗ, t_{1/2}, CL/F and V_{z}/F values are presented as arithmetic mean ± standard deviation.
tₘₐₓ and t_{lag} values are presented as median (minimum, maximum).
CuHis = Copper histidinate
^{a} = Whole blood and serum total radioactivity
^{b} = Serum [¹⁴C]-copper histidinate
^{c}In order for the descriptive statistics for whole blood and serum total radioactivity to be reflective of values calculated over the same interval of time for all subjects, Subject 6 was excluded from the descriptive statistics because his pharmacokinetic profiles for whole blood and serum total radioactivity extended to 504 hours postdose, while those of the other subjects extended only up to 168 hours postdose.
^{d}AUC_{0-∞}, AUC_{% extrap}, Kₑₗ, and t_{1/2}, could not be calculated for whole blood total radioactivity as the profiles did not exhibit a terminal elimination phase.
^{e}AUC_{0-∞}, AUC_{% extrap}, Kₑₗ, t_{1/2}, CL/F, and V_{z}/F of serum [¹⁴C]-copper histidinate could not be calculated for Subject 1 since the terminal elimination phase could not be characterized.
. = Value not reportable
NA = Not applicable

Exposure to TRA in the first 24 hours postdose (AUC₀₋₂₄) was approximately 34% lower in whole blood than in serum. By 168 hours postdose, exposure (AUC₀₋ₗₐₛₜ) to TRA was now approximately 29% higher in whole blood than in serum. Peak exposure to TRA (Cₘₐₓ) was similar in whole blood and serum although the tₘₐₓ values were different, occurring much later in whole blood (median=˜168 hours) than in serum (median=˜8 hours).

Approximately 9% of the exposure to TRA in the serum in the first 24 hours postdose was associated with [¹⁴C]-CuHis (Geom Mean AUC₀₋₂₄ Ratio=0.09317). The arithmetic mean apparent terminal t_{1/2} of serum [¹⁴C]-CuHis (˜75 hours) was shorter than that of serum TRA (˜253 hours).

### Example 6

### Juvenile Rat Toxicokinetics Study

The objectives of this study were to determine the tolerability of copper histidinate when given via twice daily subcutaneous injection to juvenile Crl:CD(SD) Sprague-Dawley rats on Postnatal Days (PND) 7 through 21 and to provide information for the selection of dose levels to be used in a subsequent juvenile toxicity study. In addition, the toxicokinetic characteristics of copper histidinate were determined.

The test article for this study is copper histidinate and is described below in TABLE 6.

TABLE 6

| | Test Article |
|---|---|
| Identification: | Copper histidinate Lyophilized Powder, |
| | 1000 μg Cu/vial |
| Alternate Identification: | CuHis; CUTX-101 |
| Physical Description: | Lyophilized blue powder, clear blue |
| | solution upon reconstitution |
| Purity: | 100% |
| Correction Factor: | 1.00 |
| Concentration: | N/A |
| Storage Conditions | −20° C. |

The vehicle control article is sodium chloride and is described below in TABLE 7.

TABLE 7

| | Vehicle Control Article |
|---|---|
| Identification: | 0.9% Sodium Chloride (NaCl) |
| | for Injection |
| Physical Description: | Clear, colorless liquid |
| Storage Conditions | Ambient temperature, Clear, |
| (temperature set to | colorless liquid |
| maintain): Physical | |
| Storage Conditions | Ambient temperature |

Animals for this study are Crl:CD(SD) Sprague Dawley rats. Phase A of the study required 6 dams (5 pups/sex/litter) and Phase B of the study required 28 dams (6 pups/sex/litter).

Number of Animals Assigned to Study—Phase A and Phase B as are shown below in Tables 8 and 9.

TABLE 8
Number of Animals Assigned to Study - Phase A

| Group No. | Males | Females |
|---|---|---|
| 1 | 3 | 3 |
| 2 | 3 | 3 |
| 3 | 3 | 3 |
| 4 | 3 | 3 |
| 5 | 3 | 3 |

| | |
|---|---|
| Total No. of Pups | 15 M + 15 F |

TABLE 9
No. of Animals Assigned to Study - Phase B

| | |
|---|---|
| Main Study | Toxicokinetic Study |

| Group No. | Males | Females | Males | Females |
|---|---|---|---|---|
| 6 | 6 | 6 | 6 | 6 |
| 7 | 6 | 6 | 39 | 39 |
| 8 | 6 | 6 | 39 | 39 |
| 9 | 6 | 6 | 39 | 39 |
| 10 | 6 | 6 | 39 | 39 |

| | | |
|---|---|---|
| Total No. of Pups | 24 M + 24 F | 123 M + 123 F |

The experimental design for Phase A is show below in TABLE 10.

TABLE 10

| | | Dose | Dose | Dose | Dose | | | |
|---|---|---|---|---|---|---|---|---|
| Group | Test | Level^{a, b, d} | Level^{a, b, d} | Concentration^{d} | Volume^{c} | Dose | No. of | No. of |
| No. | Material | (mg/kg/day) | (mg/kg/dose) | (mg/mL) | (mL/kg/dose) | Frequency | Males | Females |
| 1 | Vehicle | 0 | 0 | 0 | 1 | BID | 3 | 3 |
| | Control | | | | | | | |
| | Article | | | | | | | |
| 2 | CuHis | 0.5 | 0.5 | 0.5 | 1 | QD | 3 | 3 |
| 3 | CuHis | 10 | 5 | 5 | 1 | BID | 3 | 3 |
| 4 | CuHis | 25 | 12.5 | 5 | 2.5 | BID | 3 | 3 |
| 5 | CuHis | 20 | 10 | 5 | 2 | BID | 3 | 3 |

TBA = To be added to the Protocol by amendment.
^{a}Dose levels did not exceed 25 mg/kg/day.
^{b}Dose levels for Groups 3 through 5 were based on the results from the previous dose levels.
^{c}Based on the most recent body weight measurement.
^{d}Dose levels and concentration based on elemental copper (Cu) (1000 μg copper equivalent to 5800 μg copper histidinate).

The experimental design for Phase B (14 days) is show below in TABLE 11.

TABLE 11

| | | | | | | No. of Females | | | |
|---|---|---|---|---|---|---|---|---|---|
| | | Dose | Dose | Dose | Dose | Main | | Toxicokinetic^{b} | |
| Group | Test | Level ^{c} | Level ^{c} | Concentration | Volume^{a} | Study | | Study | |
| No. | Material | (mg/kg/day) | (mg/kg/dose) | (mg/mL) | (mL/kg) | Males | Females | Males | Females |
| 6 | Vehicle | 0 | 0 | 0 | 1.5 | 6 | 6 | 6 | 6 |
| | Control | | | | | | | | |
| 7 | CuHis | 2 | 1 | 0.67 | 1.5 | 6 | 6 | 39 | 39 |
| 8 | CuHis | 7.5 | 3.75 | 2.5 | 1.5 | 6 | 6 | 39 | 39 |
| 9 | CuHis | 10^{d} | 5^{d} | 3.33^{d} | 1.5 | 6 | 6 | 39 | 39 |

TBA = To be added to the Protocol by amendment based on the results of Phase A of the study.
^{a}Based on the most recent body weight measurement.
^{b}Toxicokinetic animals were used for toxicokinetic evaluation only.
^{c} Dose levels and concentration based on elemental Cu (1000 mcg Cu equivalent to 5800 mcg CuHis).
^{d}Dose level was reduced to 10 mg/kg/day (5 mg/kg/dose) on PND 17.

Dose Route: Subcutaneous injection. Injection sites were rotated to minimize irritation; documentation of this procedure was recorded in the raw data. Injections were made into two approximately equal areas on the dorsum (areas 1 and 2; area 1 being closest to the shoulders, area 2 closest to the tail). Each dose was injected in these areas in sequence for each animal (i.e., area 1 on the first dose, area 2 on the second dose, area 1 on the third dose, etc.).

Frequency: Phase A: Group 1 and Groups 3 to 5: Twice daily (approximately 8 hours apart) Group 2: Once daily. Phase B: All Groups: Twice daily (approximately 8 hours apart).

Duration: Phase A: PND 7 only. Phase B: PND 10 through 24.

Special Procedures: Dose administration for Phase A of the study was staggered by at least 5 days for each dose group with Groups 1 and 2 dose administration beginning on the same day.

In-life procedures, observations and measurements. For F0 generation dams, viability observations were recorded at least twice daily. Clinical observations were recorded at least once weekly. Maternal behavior (e.g., nesting, nursing) was recorded daily beginning the day of or day after arrival at the Testing Facility. Maternal body weights were recorded at least weekly and before euthanasia (terminal weight). Food consumption was not measured; however, food was monitored and replenished as necessary to monitor the health and well-being of the animals. F0 generation data were not reported.

The in-life procedures, observations, and measurements listed below were performed for F1 generation pups/rats as per Tables 8 and 9. Food consumption was not recorded for animals assigned to Phase B of the study; however, food will be monitored and replenished as necessary to monitor the health and well-being of the animals.

Phase A—General In-Life Assessments

TABLE 12

| | Frequency |
|---|---|
| Parameter | (minimum required) |
| Viability | Litters will be observed for |
| | dead pups at least twice daily |
| | until initiation of dosing. The |
| | pups in each litter will be |
| | counted at least once weekly |
| | After initiation of dosing pups |
| | will be observed for viability at |
| | least twice daily. |
| Clinical Observations: | Acclimation Period: Daily |
| General | during the acclimation period |
| | Dose Period: Daily before each |
| | administered. |
| | Postdose Period: Daily |
| | (including the day of scheduled |
| | euthanasia). |
| Clinical Observations: | 0.5 to 1 hour following each |
| Postdose Observations | dose. |
| Individual Body | Acclimation Period: On the |
| Weights | day of or day after arrival and |
| | at least once weekly until the |
| | Dose Period: Daily. |
| | Postdose Period: Daily |
| | (including the day of scheduled |
| | euthanasia). |

Phase B—General In-Life Assessments

TABLE 13

| | | Frequency |
|---|---|---|
| Parameter | Population | (minimum required) |
| Viability | Main and TK | Litters will be observed for |
| | Studies | dead pups at least twice daily |
| | | until initiation of dosing. The |
| | | pups in each litter will be |
| | | counted at least once weekly |
| | | until initiation of dosing. |
| | | After initiation of dosing |
| | | pups will be observed for |
| | | viability at least twice daily. |
| Clinical | Main and TK | Acclimation Period: Daily |
| Observations: | Studies | during the acclimation |
| General | | period |
| | | Dose Period: Daily before |
| | | each dose is administered. |
| | | Postdose Period: Daily |
| | | (including the day of |
| | | scheduled euthanasia). |
| Clinical | Main and TK | 0.5 to 1 hour following each |
| Observations: | Studies | dose. |
| Postdose | | |
| Observations | | |
| Dermal | Main Study | Prior to the first daily dose |
| Draize | | administration and 0.5 to 1 |
| Scoring of | | hour following the first daily |
| Injection Site | | dose. |
| Individual | Main and TK | Acclimation Period: On the |
| Body | Studies | day of or day after arrival |
| Weights | | and at least once weekly |
| | | until the initiation of dosing. |
| | | Dose Period: Daily. |
| | | Postdose Period: Daily |
| | | (including the day of |
| | | scheduled euthanasia). |

Bioanalytical Sample Analysis: Serum samples will be analyzed for concentration of total copper and ceruloplasmin using a qualified analytical procedure.

Toxicokinetic Evaluation: A noncompartmental approach consistent with the subcutaneous route of administration will be used for parameter estimation. All parameters will be generated from total copper and ceruloplasmin mean concentrations from PNDs 10 and 24 whenever practical.

TABLE 14
Parameters to be Estimated

| Parameter | Description of Parameter |
|---|---|
| Tₘₐₓ | The time after dosing at which the maximum concentration was observed. |
| Cₘₐₓ | The maximum observed concentration measured after dosing |
| Cₘₐₓ/Dose | The Cₘₐₓ divided by the dose administered |
| AUCTₗₐₛₜ | The area under the concentration versus time curve from the start of dose |
| | administration to the last observed quantifiable concentration calculated |
| | using the linear trapezoidal method |
| AUCTₗₐₛₜ/Dose | The AUCTₗₐₛₜ divided by the dose administered |
| AUC₀₋₈ | The area under the concentration versus time curve from the start of dose |
| | administration to 8 hours postdose calculated using the linear trapezoidal |
| | method |
| AUC₀₋₂₄ | The area under the concentration versus time curve from the start of dose |
| | administration to 24 hours postdose calculated using the linear |
| | trapezoidal method |
| R | Accumulation ratio on AUC₀₋₈ and Cₘₐₓ will be calculated as TK parameter |
| | after repeat doses divided by TK parameter during the initial dosing |
| | interval |
| Tₗₐₛₜ | The time after dosing at which the last quantifiable concentration was |
| | observed. |

Partial AUCs (between two defined sample times), and corresponding dose-normalized values, may be derived and reported to aid interpretation. Descriptive statistics (e.g., number, arithmetic mean, median, standard deviation, standard error, coefficient of variation) will be reported as deemed appropriate. TK tables and graphs will also be generated.

Although the invention has been described with reference to the above examples, it will be understood that modifications and variations are encompassed within the spirit and scope of the invention. Accordingly, the invention is limited only by the following claims.

1. A pharmaceutical composition comprising lyophilized copper histidinate and a pharmaceutically acceptable carrier.

2. The pharmaceutical composition of claim 1, wherein the pharmaceutically acceptable carrier is saline.

3. The pharmaceutical composition of claim 2, wherein the lyophilized copper histidinate is reconstituted to a concentration of about 2900 μg/ml.

4. A subcutaneous dosage form comprising copper histidinate and a pharmaceutically acceptable carrier.

5. The dosage form of claim 4, wherein the copper histidinate is reconstituted to a concentration of about 2900 μg/ml.

6. A stable lyophilized pharmaceutical formulation of copper histidinate, wherein the formulation made by lyophilizing an aqueous solution comprising:
2 moles of L-histidine and 1 mole of cupric chloride and adjusted to a pH of about 7.0 to 7.5 in an aqueous solution, followed by lyophilization of the solution.

7. The stable lyophilized pharmaceutical formulation of claim 6, wherein the copper histidinate is present at about 2900 μg/mL in the aqueous solution.

8. The stable lyophilized pharmaceutical formulation of claim 6, wherein the formulation is stable for about 18 months at about −10 to −20° C.; 2 to 8° C.; or room temperature (15 to 30° C.).

9. A vial comprising the formulation of claim 6, and a pharmaceutically acceptable carrier.

10. The vial of claim 9, wherein the pharmaceutically acceptable carrier is saline.

11. The vial of claim 10, wherein the formulation is reconstituted in 2 ml of saline to a concentration of about 2900 μg/ml.

12. The vial of claim 10, wherein the formulation comprises lyophilized copper histidinate reconstituted in saline at a concentration of 500 μg elemental copper/ml in the vial.

13. The vial of claim 9, further comprising instructions or a label for reconstitution of the lyophilized copper histidinate in saline and subcutaneous administration to a subject with Menkes disease or a copper deficiency at a dose of 0.5 ml per administration twice daily until the subject is one year of age and once daily after the subject is one year of age.

14. The vial of claim 9, wherein the reconstituted lyophilized copper histidinate is stable at room temperature for at least 24 hours or at 2 to 8° C. for at least about 7 days.

15. The vial of claim 13, wherein the subject with Menkes has an ATP7A mutation selected from a nonsense variant, missense variant, intron variant, synonymous variant, splice region variant, frameshift variant, splice acceptor variant, 5′ UTR variant, splice donor variant, duplicative variant, in frame deletion, gross deletion, chromosomal translocation, canonical splice site mutation or a combination thereof.

16. The vial of claim 13, wherein the instructions include performing a genetic test to identify an ATP7A mutation in the subject.

17. A container comprising the formulation of claim 6, and a pharmaceutically acceptable carrier.

18. The container of claim 17, wherein the carrier is saline.

19. The container of claim 17, wherein the lyophilized formulation and the saline are separated in the container.

20. The container of claim 17, wherein the container comprises two chambers for mixing the formulation with the carrier to form a mixed formulation prior to injecting the mixed formulation into a subject.

21. The container of claim 20, wherein the container is a syringe, an ampoule or a vial.

22-32. (canceled)

33. A method of increasing serum copper histidinate levels in a subject comprising administering copper histidinate to the subject via a subcutaneous route of administration with a peak detectable level of copper histidinate in the serum at about ½ hour to 1 hour following administration, thereby increasing serum copper histidinate levels in the subject.

34-39. (canceled)

40. The method of claim 33, wherein the subject has Menkes disease or other copper deficiencies.
